SEQUENCE LISTING (7 sequences)

SEQ ID NO: 1 - protein, 577 aa, Homo sapiens
MPKTISVRVTTMDAELEFAIQPNTTGKQLFDQVVKTIGLREVWFFGLQYQDTKGFSTWLKLNKKVTAQDVRKESPLLFKFRAKFYPEDVSEELIQDITQRLFFLQVKEGILNDDIYCPPETAVLLASYAVQSKYGDFNKEVHKSGYLAGDKLLPQRVLEQHKLNKDQWEERIQVWHEEHRGMLREDAVLEYLKIAQDLEMYGVNYFSIKNKKGSELWLGVDALGLNIYEQNDRLTPKIGFPWSEIRNISFNDKKFVIKPIDKKAPDFVFYAPRLRINKRILALCMGNHELYMRRRKPDTIEVQQMKAQAREEKHQKQMERAMLENEKKKREMAEKEKEKIEREKEELMERLKQIEEQTKKAQQELEEQTRRALELEQERKRAQSEAEKLAKERQEAEEAKEALLQASRDQKKTQEQLALEMAELTARISQLEMARQKKESEAVEWQQKAQMVQEDLEKTRAELKTAMSTPHVAEPAENEQDEQDENGAEASADLRADAMAKDRSEEERTTEAEKNERVQKHLKALTSELANARDESKKTANDMIHAENMRLGRDKYKTLRQIRQGNTKQRIDEFESM

SEQ ID NO: 2 - DNA, 17 bp, Artificial Sequence
  PCR Primer (Forward HER2)
gccgcagtgagcaccat

SEQ ID NO: 3 - DNA, 19 bp, Artificial Sequence
  PCR Primer (Reverse HER2)
cgcagcttcatgtctgtgc

SEQ ID NO: 4 - DNA, 27 bp, Artificial Sequence
  PCR Primer (Forward MSN)
ggaatgagacaggacctaggatatctt

SEQ ID NO: 5 - DNA, 27 bp, Artificial Sequence
  PCR Primer (Reverse MSN)
ggaatgagacaggacctaggatatctt

SEQ ID NO: 6 - DNA, 21 bp, Artificial Sequence
  PCR Primer (Forward GAPDH)
gatccctccaaaatcaagtgg

SEQ ID NO: 7 - DNA, 22 bp, Artificial Sequence
  PCR Primer (Reverse GAPDH)
gcaaatgagccccagccttctc

(19) United States
(12) Patent Application Publication
(10) Pub. No.: US 2020/0308652 A1
(43) Pub. Date: Oct. 1, 2020

(54) DIAGNOSIS AND/OR PROGNOSIS OF HER2-DEPENDENT CANCER USING MOESIN AS A BIOMARKER

(71) Applicants: UNIVERSITE PARIS DESCARTES, PARIS Cedex 06 (FR); INSTITUT NATIONAL DE LA SANTE ET DE LA RECHERCHE MEDICALE (INSERM), PARIS Cedex 13 (FR); CENTRE NATIONAL DE LA RECHERCHE SCIENTIFIQUE (C.N.R.S), PARIS Cedex 16 (FR)

(72) Inventors: Sandrine BOURDOULOUS, Gagny (FR); Anaïs DOMINGOT, Paris (FR); Camille FAURE, Paris (FR)

(21) Appl. No.: 16/758,644

(22) PCT Filed: Oct. 24, 2018
(86) PCT No.: PCT/EP2018/079213

§ 371 Date: Apr. 23, 2020

(30) Foreign Application Priority Data
Oct. 24, 2017 (EP) 17306465.0

(51) Int. Cl. C12Q 1/6886

(52) U.S. Cl. CPC C12Q 1/6886; G01N 2800/52; C12Q 2600/158; C12Q 2600/118

(57) ABSTRACT

This invention relates to a method for diagnosing and/or prognosticating HER2-dependent cancer in a subject, comprising a) measuring the amount of moesin in a sample from the subject; b) comparing the amount of moesin measured in step a) to a reference value; c) finding a deviation or no deviation of the amount of moesin measured in step a) from the reference value; and d) attributing said finding of deviation or no deviation to a particular diagnosis and/or prognosis of HER2-dependent cancer in the subject.

### BACKGROUND OF THE INVENTION

Breast cancer is the most common cancer in women worldwide, with nearly 1.7 million new cases diagnosed in 2012 (second most common cancer overall). This represents about 12% of all new cancer cases and 25% of all cancers in women. About half the breast cancer cases and 60% of the deaths are estimated to occur in economically developing countries. The rate of incidence observed in France is among the strongest in Europe and is in constant increase.

About 20 to 30% of primary human breast cancers are due to the deregulated expression of HER2: it represents approximately 8,000 patients a year in France and 450,000 patients a year worldwide. HER2 is a well-known member of the human epidermal growth factor receptor (HER/EGFR/ERBB) family. ErbB2 overexpression is associated with a poor diagnosis: tumors with deregulated HER2 have been shown to grow faster to be more aggressive and to be less sensitive to chemotherapy or to hormonotherapy. HER2 deregulation is also associated with disease recurrence. Then, so called HER2-dependent cancer constitute a very specific group of cancer of an utmost interest in public health. Indeed, only 25% of the treated patients respond to the actual therapies. The actual strategies aiming at targeting the extracellular domain of HER2 (anti-HER2 antibody therapies such as Herceptin/Trastuzumab, Pertuzumab and the recently developed Trastuzumab emtansine from Genentech, USA) or the kinase activity of the receptor (small molecule tyrosine kinase inhibitors, such as Lapatinib/Tykerb, GSK, USA) have proven to exhibit limited actions.

In particular, these molecules have no potent action on the mutated and truncated forms of HER2. Concerning Trastuzumab, 66% to 88% of treated patients never respond to treatment (i.e. present a “primary resistance”) and among the one-third of the treated patients that respond to this agent, a disease progression on average in less than one year (i.e. develop an “acquired resistance”) is generally observed. The efficacy is limited by the development of therapeutic resistance mainly attributed to the expression of p95HER-2, as this highly active truncated form of HER2 lacks the recognition site for Trastuzumab.

Generally, HER2 (over)expression is established as a marker of poor prognosis in breast cancer. Different techniques have been developed to evaluate if a cancer/patient is “positive” for HER2 (i.e. HER2+) or “negative” for HER2 (i.e. HER2−), such as fluorescence in situ hybridization (FISH) test or immunohistochemistry (IHC test).

The inventors have identified a strong functional link between moesin and HER2 genes and found that that diagnosis of HER2-dependent cancer or a poor prognosis of HER2-dependent cancer is specifically associated with a low level of moesin in HER2+ subjects.

This finding makes it possible to develop a new method for diagnosing and/or prognosticating HER2-dependent cancer in a subject.

### SUMMARY OF THE INVENTION

The inventors therefore propose here a method for the diagnostic and/or prognosis wherein the amount of moesin in the sample from a subject compared to a reference value represents the prediction of HER2-dependent cancer or represents a prognosis for HER2-dependent cancer, indicating that the subject has a risk or no of having and/or developing HER2-dependent cancer.

The invention relates to a method for diagnosing and/or prognosticating HER2-dependent cancer in a subject, comprising:
a) measuring the amount of moesin in a sample from the subject;
b) comparing the amount of moesin measured in step a) to a reference value;
c) finding a deviation or no deviation of the amount of moesin measured in step a) from the reference value; and
d) attributing said finding of deviation or no deviation to a particular diagnosis and/or prognosis of HER2-dependent cancer in the subject.

The invention also relates to a method for monitoring a change in the diagnosis and/or prognosis of HER2-dependent cancer in a subject, comprising:
a) applying the method of the invention to the subject at one or more successive time points, whereby the diagnosis and/or prognosis of HER2-dependent cancer in the subject is determined at said successive time points;
b) comparing the diagnosis and/or prognosis of HER2-dependent cancer in the subject at said successive time points as determined in step a); and
c) finding the presence or absence of a change between the diagnosis of HER2-dependent cancer in the subject at said successive time points as determined in step a).

The invention also relates to a kit for determining the diagnostic and/or the prognosis of HER2-dependent cancer in a subject suffering thereof, comprising:
means for measuring the amount of moesin in the sample from the subject; and
a reference value of the amount of moesin or means for establishing said reference value, said reference value representing a known diagnosis and/or prognosis of HER2-dependent cancer.

The invention also relates to the use of a kit comprising:
means for measuring the amount of moesin in the sample from the subject; and
a reference value of the amount of moesin or means for establishing said reference value, said reference value representing a known diagnosis and/or prognosis of HER2-dependent cancer

for determining the diagnostic and/or the prognosis of HER2-dependent cancer in a subject suffering thereof.

The invention also relates to the use of the two probes SEQ ID NO: 4 and SEQ ID NO: 5 for measuring the mRNA level of moesin by qRT-PCR for determining the diagnostic and/or the prognosis of HER2-dependent cancer in a human subject suffering thereof.

### DETAILED DESCRIPTION OF THE INVENTION

### Definitions

The terms “diagnosing” or “diagnosis” and “prognosticating” or “prognosis”, as used herein, are used in the broadest sense, and are commonly used and are well-understood in medical and clinical practice. By means of further explanation and without limitation, “prognosticating” or “prognosis” refers to the determination of probability, risk or possibility of developing HER2-dependent cancer in a subject. By means of further explanation and without limitation, “diagnosing” or “diagnosis” refers to the determination of a probability or a possibility of having HER2-dependent cancer in a subject.

The term “HER2”, as used herein, refers to “Human Epidermal growth factor Receptor 2” which is a protein member of the human epidermal growth factor receptor family. “HER2” is also frequently called “ErbB2”. “ErbB2” and “HER2” are used interchangeably in the present invention.

The term “HER2-dependent cancer” or “HER2 positive cancer” or “HER2+ cancer”, as used herein, refers to cancer involving exacerbated HER2 activation. In particular, the term “HER2-dependant cancer” refers to any cancer case for which cancer cells exhibiting a deregulation of HER2 gene have been identified, in opposition to “HER2-independent cancer” or “HER2 negative cancer” or “HER2− cancer”. More particularly said deregulation can correspond to an amplification of HER2 gene. This amplification can be detected at the genetic level, or at the protein level. For example, guidelines emitted by the American society of Clinical Oncology/College of American Pathologists (ASCO/CAP) for breast cancer set several cut-offs for determining the ErbB2 status of breast cancer. These guidelines prescribe that a cancer should be considered as “HER2-dependent” if, for the primary site and if possible for the metastatic site:
a uniform and intense membrane staining of more than 30% of invasive tumor cells is observed in immunochemistry (IHC), or
i) a FISH amplified ratio of HER2 to CEP17 (chromosome 17 centromere) superior or equal to 2 (dual probe testing) or ii) a FISH amplified ratio of HER2 to CEP17 (chromosome 17 centromere) inferior to 2 (dual probe testing) with an average HER2 copy number of at least 6 copies per nucleus (single probe testing) is determined, or iii) a single probe average of at least 6 signals for the HER2 copy number per cells.

Besides, a cancer is considered as “HER2-independent” when, for the primary site and if possible for the metastatic site:
in IHC, no staining or a weak incomplete membrane staining, or a weak but complete membrane staining is observed in less than 10% of cells, or
the FISH HER2/CEP17 ratio inferior to 2 with an average copy number of HER2 inferior to 4 signals per cells is noticed (dual probe testing), or an average copy number of HER2 inferior to 4 signals per cell is noticed (in cases where a single probe is used).

The HER2 status will be considered as equivocal (then a new test should be performed) when, for the primary site and, if possible, for the metastatic site:
in IHC, i) an incomplete labelling of circumferential membrane and/or weak/moderate labelling is noticed but within superior to 10% of the invasive tumor cells or ii) a complete and intense labelling of circumferential membrane is noticed but for 10% or less of the invasive tumor cells, or
the FISH HER2/CEP17 ratio inferior to 2 with an average copy number of HER2 of at least 4 signals but less than 6 signals per cell is noticed (dual probe testing), or an average copy number of HER2 of at least 4 signals but less than 6 signals per cell is noticed (in cases where a single probe is used).

Deregulation of HER2 gene can also correspond to activating mutations in HER2 gene disregarding its copy number, leading to an increase of the tyrosine kinase activity of HER2. For example, said activating mutations can be V659E, G309A, D769H, D769Y, V777L, P780ins, V842I, R896C, K753E or L755S and can be detected by Polymerase Chain Reaction or any sequencing technique [Bose et al. Cancer Discov. 2013, 3(2), 224-237; Zuo et al. Clin Cancer Res 2016, 22(19), 4859-4869]. Also, both an amplification of HER2 gene and a somatic activating mutation can be detected in the same case of cancer.

Well known molecular biology tests other than Fish or IHC, using negative and positive control cells with an established HER2 status, can be used for determining the HER2 status of a cancer by way of comparison, as for example Enzyme-Linked Immunosorbent Assays, Western blotting assays, Polymerase Chain Reaction, etc.

Preferably, the HER2-dependent cancer according to the invention is selected from the group consisting of breast cancer, female genital tract cancer, such as endometrial cancer, uterine cancer or ovarian cancer, bladder cancer, anal cancer, colorectal cancer, in particular uterine serous cancer, such as uterine papillary serous carcinoma, lung cancer, in particular non-small-cell lung cancer, liver cancer, kidney cancer, gastroesophageal cancer, stomach cancer, pancreas cancer and gastric cancer. In a preferred embodiment, the HER2-dependent cancer may be HER2+ breast cancer, HER2+ ovarian cancer, HER2+ bladder cancer, HER2+ colorectal cancer, HER2+ uterine papillary serous carcinoma and HER2+ gastric cancer, preferably HER2+ breast cancer.

For the purposes of the invention, the term “marker” (or “biomarker”) is an entity, such as a protein or a ribonucleic acid (RNA), which can be used for the diagnosis and/or prognosis of HER2-dependent cancer. According to the invention, the biomarker is moesin, i.e. the protein itself or a RNA coding said moesin, such as a mRNA. For the purposes of the invention, the biomarker is preferably a protein.

Moesin (for membrane-organizing extension spike protein) is a protein that in humans is encoded by the MSN gene. Moesin is a member of the ERM protein family which consists of three closely related proteins, Ezrin, Radixin and Moesin, that play a key role in a large number of important physiological processes such as cell morphogenesis, adhesion or migration, as they provide a regulated linkage between membrane proteins and the cortical cytoskeleton and trigger signal-transduction pathways. Exemplary moesin includes, without limitation, human moesin having the amino acid sequence SEQ ID NO: 1 (Uniprot accession number: P26038).

The term “subject” or “patient”, as used herein, refers to a human or non-human mammal (such as a rodent (mouse, rat), a feline, a canine, or a primate) affected or likely to be affected with HER2-dependent cancer. Preferably, the subject is a human, man or woman. Preferably, the subject is HER2+.

The term “HER2+ subject”, as used herein, refers to a subject having a cancer being positive for the expression and/or activation of HER2. Different techniques may be used for assaying HER2 expression, for example fluorescence in situ hybridization (FISH) test, chromogenic in situ hybridization, real-time quantitative polymerase-chain-reaction (qRT-PCR) or immunohistochemistry (IHC) test.

The term “sample” or “biological sample”, as used herein, includes any biological specimen obtained from a subject, such as a tumor sample or a body fluid sample. A body fluid sample may be selected from the group consisting of blood, serum plasma, saliva, urine, amniotic fluid, breast milk, bronchial lavage, cerebrospinal fluid, peritoneal fluid, seminal fluid, tears and pleural fluid, preferably blood. A tumor sample may include biopsies, such as tumor biopsies.

The term “reference value” or “reference profile” means a value or profile representing (i) a diagnosis of no HER2-dependent cancer, (ii) a diagnosis of HER2-dependent cancer, (iii) a good prognosis of HER2-dependent cancer or (iv) a bad prognosis of HER2-dependent cancer. For example, a reference value for the amount of moesin may be obtained as follows:
a) measuring the amount of moesin in:
   a1) one or more samples from one or more subjects not having HER2-dependent cancer, or
   a2) one or more samples from one or more subjects having HER2-dependent cancer, and
b) storing the quantity of moesin
   b1) as measured in (a1) as the reference value representing the diagnosis and/or prognosis of no HER2-dependent cancer, or
   b2) as measured in (a2) as the reference value representing the diagnosis and/or prognosis of HER2-dependent cancer.

A reference value also encompasses a reference range.

The terms “amount”, “quantity”, and “level” are synonymous and generally well-understood in the art. The terms as used herein may particularly refer to an absolute quantification of a biomarker in a sample, or to a relative quantification of a biomarker in a sample (i.e. relative to another value such as relative to a reference value as taught herein), or to a range of values indicating a base-line expression of the biomarker. These values or ranges can be obtained from a single patient or from a group of patients. An absolute quantity of a biomarker in a sample may be advantageously expressed as weight or as molar amount, or more commonly as a concentration. A relative quantity of a biomarker in a sample may be advantageously expressed as an increase or a decrease relative to (i.e. compared to) another value, such as relative to (i.e. compared to) a reference value.

The term “deviation” of a first value from a second value may generally encompass any direction, such as increase or decrease of a first value compared to a second value.

The term “treating” or “treatment” means reversing, alleviating, inhibiting the progress of, or preventing the disorder or condition to which such term applies, or one or more symptoms of such disorder or condition. In particular, the treatment of the disorder may consist in destroying, depleting or inhibiting the proliferation of cancer cells. Most preferably, such treatment leads to the complete depletion of cancer cells.

Diagnosis and/or Prognosis

The present invention results from the advantages highlighted by the inventors that the increase or decrease of the amount of moesin makes it possible to diagnose and/or prognose HER2-dependent cancer. The resulting applications are described below.

The invention relates to a method for diagnosing and/or prognosticating HER2-dependent cancer in a subject, comprising:
a) measuring the amount of moesin in a sample from the subject;
b) comparing the amount of moesin measured in step a) to a reference value;
c) finding a deviation or no deviation of the amount of moesin measured in step a) from the reference value; and
d) attributing said finding of deviation or no deviation to a particular diagnosis and/or prognosis of HER2-dependent cancer in the subject.

The methods of the invention may be used in individuals who have not yet been diagnosed as having HER2-dependent cancer (for example, preventative screening), or who have been diagnosed as having HER2-dependent cancer, or who are suspected of having HER2-dependent cancer (for example, display one or more symptoms characteristic of HER2-dependent cancer), or who are at risk of developing HER2-dependent cancer. The methods may also be used to detect various stages of progression or severity of HER2-dependent cancer.

Step a)

Step a) is the measure of the amount of moesin in a sample from the subject.

The amount of moesin may be measured by any suitable technique known in the prior art. In some embodiments, the amount of moesin a may be measured using a binding agent capable of specifically binding to moesin and/or to fragments thereof. The binding agent may be an antibody, an aptamer, a protein, a peptide or a small molecule. For example, the amount of moesin is measured using an immunoassay technology, such as enzyme-linked immunosorbent assay (ELISA), Immunoradiometric Assay (IRMA) or radioimmunoassay (RIA), or using a mass spectrometry analysis method or using a chromatography method, or using a combination of said methods. In some other embodiments, the amount of moesin may be determined by the measure of the moesin mRNA using any suitable technique, such as qRT-PCR or digital molecular barcoding technology for RNA or DNA microarrays.

Step b) & c)

Step b) and c) is the comparison of the amount of moesin measured in step a) to a reference value and the finding of a deviation or no deviation of the amount of moesin measured in step a) from the reference value.

A deviation of the amount of moesin may encompass a decrease of the amount of moesin compared to the reference value or an increase of the amount of moesin compared to the reference value.

For example, a deviation of the amount of moesin compared to the reference value may encompass a decrease of at least about 10% (about 0.9-fold or less), or by at least about 20% (about 0.8-fold or less), or by at least about 30% (about 0.7-fold or less), or by at least about 40% (about 0.6-fold or less), or by at least about 50% (about 0.5-fold or less), or by at least about 60% (about 0.4-fold or less), or by at least about 70% (about 0.3-fold or less), or by at least about 80% (about 0.2-fold or less), or by at least about 90% (about 0.1-fold or less), or more compared to the reference value.

For example, a deviation of the amount of moesin compared to the reference value may encompass an increase of at least about 10% (about 1.1-fold or more), or by at least about 20% (about 1.2-fold or more), or by at least about 30% (about 1.3-fold or more), or by at least about 40% (about 1.4-fold or more), or by at least about 50% (about 1.5-fold or more), or by at least about 60% (about 1.6-fold or more), or by at least about 70% (about 1.7-fold or more), or by at least about 80% (about 1.8-fold or more), or by at least about 90% (about 1.9-fold or more), or by at least about 100% (about 2-fold or more), or by at least about 150% (about 2.5-fold or more), or by at least about 200% (about 3-fold or more), or by at least about 500% (about 6-fold or more), or by at least about 900% (about 10-fold or more), or more, compared to a reference value.

In a preferred embodiment, the amount of the moesin in the sample from the subject is at least 0.5-fold decreased compared to the reference value, more preferably at least 0.7-fold decreased.

In another preferred embodiment, the amount of moesin in the sample from the subject is at least 1.5-fold increased compared to the reference value, more preferably at least 2-fold increased.

In another preferred embodiment, the amount of moesin in the sample from the subject is equal to the reference value.

Step d)

Step d) is the attribution of the finding of deviation or no deviation in step c) to a particular diagnosis and/or prognosis of HER2-dependent cancer in the subject.

A deviation which is an increase of the amount of moesin compared to the reference value represents the diagnosis of no HER2-dependent cancer or represents a good prognosis for HER2-dependent cancer. Accordingly, a higher amount of moesin in the sample from the subject compared to the reference value indicates that the subject has no HER2-dependent cancer or indicates a long-term survival of said subject.

A deviation which is a decrease of the amount of moesin compared to the reference value represents the diagnosis of HER2-dependent cancer or represents a bad prognosis for HER2-dependent cancer. Accordingly, a lower amount of moesin in the sample from the subject compared to the reference value indicates that the subject has HER2-dependent cancer and/or indicates a short-term survival of said subject.

When no deviation is found between the amount of moesin and a reference value, the absence of such deviation is indicative of or may be attributed to the conclusion that the diagnosis and/or prognosis of HER2-dependent cancer is substantially the same as that represented by the reference value.

The methods of the invention may also be combined with the assessment of one or more further biomarkers relevant for HER2-dependent cancer.

The one or more biomarkers may be chosen from the group consisting of ER/PR (Estrogen Receptor/Progesterone Receptor), HER2, p95HER2, KI67, TOP2A (Topoisomerase 2-alpha), BCL2 (B-cell lymphoma 2), GSTM1 (Glutathione S-Transferase Mu 1), BAG1, Cyclin B1, cathepsin L2, CD68 and TP53.

A Method for Monitoring a Change in the Diagnosis and/or Prognosis

The method for diagnosing and/or prognosticating HER2-dependent cancer in a subject may also be used to detect response of HER2-dependent cancer to prophylactic or therapeutic treatments or other interventions, e.g. by performing the methods at different time points during said prophylactic or therapeutic treatment or other intervention.

The invention therefore relates to a method for monitoring a change in the diagnosis and/or prognosis of HER2-dependent cancer in a subject, comprising:
a) applying the method of any of claims 1 to 10 to the subject at one or more successive time points, whereby the diagnosis and/or prognosis of HER2-dependent cancer in the subject is determined at said successive time points;
b) comparing the diagnosis and/or prognosis of HER2-dependent cancer in the subject at said successive time points as determined in step a); and
c) finding the presence or absence of a change between the diagnosis of HER2-dependent cancer in the subject at said successive time points as determined in step a).

In some embodiments, said change in the diagnosis and/or prognosis of HER2-dependent cancer in the subject is monitored in the course of a medical treatment of said subject.

The medical treatment may be a prophylactic treatment or a therapeutic treatment, preferably an antitumoral treatment. The antitumoral treatment may be any treatment known for the treatment of said HER2-dependent cancer. For example, the antitumoral treatment may aim at targeting the extracellular domain of HER2, such as anti-HER2 antibody therapies, for example Herceptin/Trastuzumab, Pertuzumab and Trastuzumab emtansine (Genentech, USA); or the antitumoral treatment may aim at targeting the kinase activity of HER2, such as small molecule tyrosine kinase inhibitors, for example, Neratinib, Tucatinib or, Lapatinib/Tykerb (GSK, USA). The antitumoral treatment may also be a taxane-based chemotherapy, such as paclitaxel.

Kit

The invention also relates to a kit for determining the diagnostic and/or the prognosis of HER2-dependent cancer in a subject suffering thereof, comprising:
means for measuring the amount of moesin in the sample from the subject; and
a reference value of the amount of moesin or means for establishing said reference value, said reference value representing a known diagnosis and/or prognosis of HER2-dependent cancer.

The invention also relates to the use of a kit comprising:
means for measuring the amount of moesin in the sample from the subject; and
a reference value of the amount of moesin or means for establishing said reference value, said reference value representing a known diagnosis and/or prognosis of HER2-dependent cancer

for determining the diagnostic and/or the prognosis of HER2-dependent cancer in a subject suffering thereof.

In some embodiments, means may comprise a binding agent capable of specifically binding to moesin and/or to fragments thereof. The binding agent may be an antibody, an aptamer, a protein, a peptide or a small molecule.

In some other embodiments, means may comprise specific amplification primers and/or probes for the specific quantitative amplification of moesin mRNA, for example the primers and/or probes are SEQ ID NO: 4 and SEQ ID NO: 5.

The invention also relates to the use of primers and/or probes SEQ ID NO: 4 and SEQ ID NO: 5 for the specific quantitative amplification of moesin mRNA for determining the diagnostic and/or the prognosis of HER2-dependent cancer in a human subject suffering thereof.

Method of Treatment

As described above, the present invention is useful to detect response of HER2-dependent cancer to prophylactic or therapeutic treatments or other interventions, e.g. by performing the methods at different time points during said prophylactic or therapeutic treatment or other intervention.

Similarly, the present invention also relates to a method for treating HER2-dependent cancer in a subject, comprising:

(i) the implementation of a diagnostic or prognostic method according to the invention, and

(ii) when the method indicates that the subject has HER2-dependent cancer and/or indicates a short-term survival of said subject, institute appropriate treatment in said subject.

In one particular embodiment, said appropriate treatment is one or more medicines selected from a prophylactic treatment or a therapeutic treatment, preferably an antitumoral treatment aiming at targeting the extracellular domain of HER2, such as anti-HER2 antibody therapies, for example Herceptin/Trastuzumab, Pertuzumab and Trastuzumab emtansine (Genentech, USA) or aiming at targeting the kinase activity of HER2, such as small molecule tyrosine kinase inhibitors, for example Neratinib, Tucatinib or Lapatinib/Tykerb (GSK, USA).

The following examples illustrate the present invention without limiting the scope of the invention to said examples.

### FIGURES

### Brief Summary of the Figures

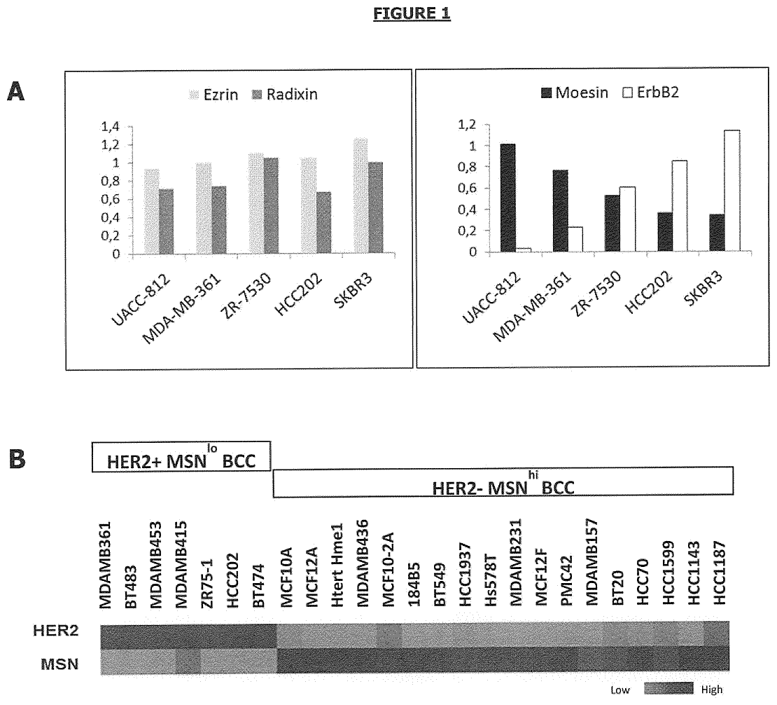

FIG. 1 represents the quantification of western blot analyses of the level of expression of Ezrin and Radixin (FIG. 1A, left panel) and HER2 and moesin (FIG. 1A, right panel) in some breast cancer cell lines with different HER2 status. FIG. 1a shows a strong inverse correlation between expression/activation levels of HER2 and expression levels of Moesin. This result was further confirmed at the mRNA level by qRT-PCR analysis performed on 25 breast cancer cells lines with various HER2 status (FIG. 1B).

MSN^{hi} means high expression of MSN.

MSN^{lo} means low expression of MSN.

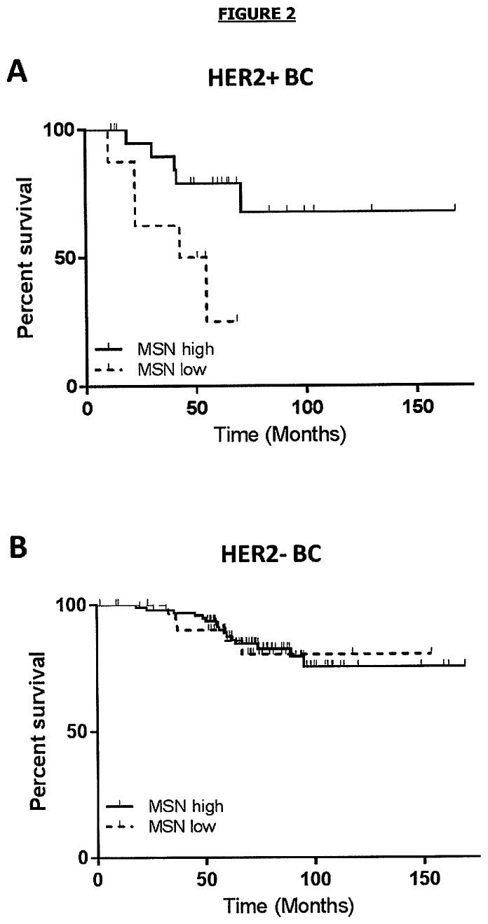

FIG. 2 represents Kaplan-Meier overall survival analyses of 30 patients with HER2+ breast cancer (HER2+ BC) or 130 patients with HER2− breast cancer (HER2− BC) depending on moesin level of expression. BC means “Breast Cancer”.

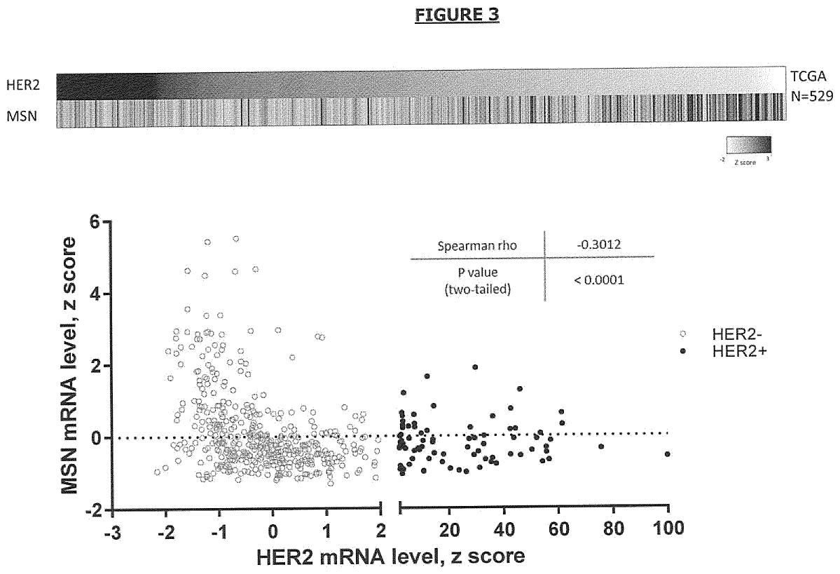

FIG. 3 represents the inverse correlation between the expression of HER2 and MSN mRNA data (Agilent microarray) from 529 tumor samples from patients with breast invasive carcinoma of TCGA cohort.

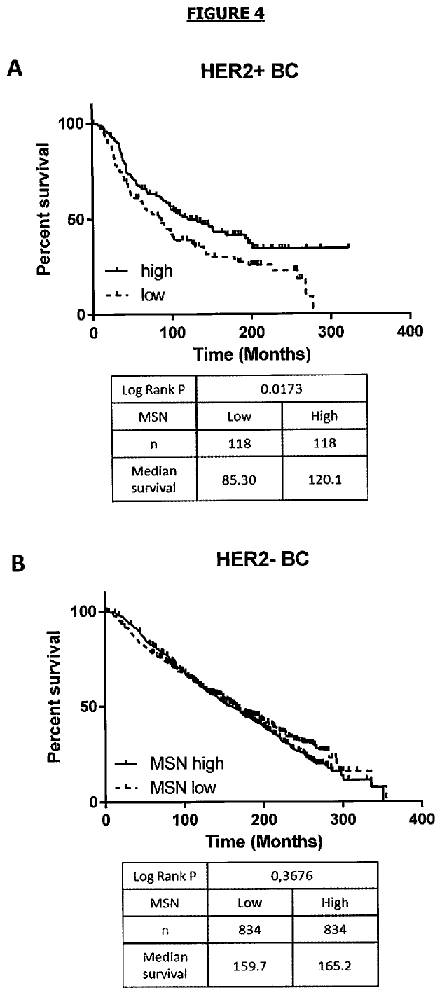

FIG. 4 represents Kaplan-Meier overall survival analyses of patients from METABRIC cohort with HER2+ breast cancer (n=236, HER2+ BC) or HER2− breast cancer (n=1668, HER2− BC) depending on moesin level of expression.

### EXAMPLES

### Example 1: Methods

Methods:

Datasets and mRNA Expression Analysis

MSN and HER2 mRNA expression profiles in 529 samples from patients with breast invasive carcinoma of TCGA cohort were collected from cbioportal database (http://www.cbioportal.org). A color-coded representation of their expression is shown in the top panel whereas a scatter plot is shown in the bottom panel. Correlation analysis was then done by Spearman rho coefficient calculation using GraphPad prism software (FIG. 3).

Datasets and mRNA Expression Analyses

Overall survival of breast cancer patients was assessed with the online Kaplan-Meier plotter (http://kmplot.com/analysis/). Patients (Gene expression data and survival information originated from 1,809 patients from GEO (Affymetrix HGU133A and HGU133+2 microarrays) were divided into two groups according to lower quartile of moesin expression levels. Analyses were either restricted to HER2 positive status ((ICH) only and molecular subtype HER2+ER, n=30) or to HER2− (ER+/−, n=130) breast cancer patients. The two patient cohorts are compared by a Kaplan-Meier survival plot, and the hazard ratio with 95% confidence intervals and log rank P value are calculated. Each database is updated biannually (FIG. 2).

Overall survival of breast cancer patients from METABRIC cohort was also analyzed based on their clinical and transcriptomic profiles obtained in the cbioportal database (http://www.cbioportal.org/). 236 HER2+ breast cancer patients or 1668 HER2− breast cancer patients were divided into two groups according to median moesin expression levels. The two patient cohorts are compared by a Kaplan-Meier survival plot, and the hazard ratio with 95% confidence intervals and Log-rank (Mantel-Cox) test value were calculated.

Cell Lines

Lysates from breast cancer cell line with different HER2 status were analyzed UACC-812 (ATCC® CRL-1897™), MDA-MB-361 (ATCC® HTB-27™), ZR-75-30 (ATCC® CRL-1504™), HCC202 (ATCC® CRL-2316™) and SK-BR-3 [SKBR3] (ATCC® HTB-30™) and were obtained from Dr. Emmanuelle Charafe-Jauffret (Institut Paoli-Calmettes, Aix Marseille University).

Reagents

Primary antibodies used were anti-HER2 (ab2428, Abcam; 1:1000), anti-clathrin (BD Biosciences #610500; 1:1000). Secondary antibodies were anti-rabbit (# NA9340-1ML GE Healthcare) or mouse IgG (# NA9310-1ML GE Healthcare) coupled with HRP (GE Healthcare; 1:5000). The anti-MSN was a gift from Dr. Paul Mangeat (Universit\tilde{e} de Montpellier) and was used at a 1:1000 dilution.

Western Blot

Lysates were denaturated with Laemmli buffer 10 min at 95° C. and proteins were separated on 10% SDS/polyacrylamide gels (ProSieve, Lonza) in gradient buffer (100 mM Tris, 100 mM Tricine, 0.1% SDS), and transferred onto nitrocellulose membranes (Whatman®) using transfert buffer (20% EtOH, 25 mM Tris, 192 mM Glycine pH 8.5) according to standard protocols. Membranes were blocked using 3% BSA in TBST buffer (140 mM NaCl, 3 mM KCl, 25 mM Tris, 0.1% Tween) 30 min at room temperature, and incubated over night at 4° C. with primary antibodies. Horseradish peroxidase-conjugated anti-rabbit or anti-mouse antibody was used as a secondary antibody. Detection was performed using the SuperSignal West Pico Chemiluminescent Substrate (Pierce) on the FUSION-FX7—SPECTRA acquisition system (Vilber Lourmat).

RNA Extraction, cDNA Preparation and Quantitative Real Time RT-PCR Analysis

Total RNA was extracted from cells using Tri Reagent® (Sigma T9424). The concentration and integrity of total RNA were measured using a NanoDrop 2000 spectrophotometer (Thermo Scientific, Wilmington, Del.). Reverse transcription reactions were performed with 200 ng total RNA using SuperScript™ II RT after RNaseOUT™ treatment (Thermo Scientific, Wilmington, Del.). Real time quantitative PCR (qRT-PCR) was performed in 10 μl using SYBR Green I Master kit (Roche Life Science) according to the manufacturer's instructions on a LightCycler 480 Instrument II.

The PCR primers were:

HER2
Forward HER2
(SEQ ID NO: 2)
5′-GCCGCAGTGAGCACCAT-3
Reverse HER2
(SEQ ID NO: 3)
5′-CGCAGCTTCATGTCTGTGC-3′
MSN
Forward MSN
(SEQ ID NO: 4)
5′-GGAATGAGACAGGACCTAGGATATCTT-3′
Reverse MSN
(SEQ ID NO: 5)
5′-GGAATGAGACAGGACCTAGGATATCTT-3′
GAPDH
Forward GAPDH
(SEQ ID NO: 6)
5′-GATCCCTCCAAAATCAAGTGG-3′
Reverse GAPDH
(SEQ ID NO: 7)
5′-GCAAATGAGCCCCAGCCTTCTC-3′

The PCR conditions were: 95° C., 10 s; 60° C., 10 s; 72° C., 15 s. At the conclusion of each run, a melt curve analysis was performed to ensure that a single product had been synthesized. The relative expression of HER2, normalized to GAPDH, was calculated using the 2-ΔΔCt method. The HER2 and MSN mRNA level analysis performed on 25 breast cancer cell lines with various HER2 status.

### Example 2: Influence of Moesin Expression on HER2 Expression

Materials and Methods

HER2 and moesin expression levels were analyzed by western blot and quantified in some breast cancer cell lines with different HER2 status (UACC812, MDAMB361, ZR-7530, HCC202 and SKBR3) (FIG. 1A).

HER2 and moesin mRNA expression levels were obtained from agilent microarray data and collected from 529 patients with breast invasive carcinoma of TCGA cohort (http://www.cbioportal.org). A color-coded representation of their expression is shown in the top panel whereas a scatter plot is shown in the bottom panel. Patients with low level of HER2 expression (zscore<2) are shown in light grey whereas patients exhibiting high level of HER2 expression (zscore≥2) are shown in black. Correlation between HER2 and MSN mRNA expression was then assessed by Spearman rho coefficient calculation (r=−0.3012, P<0.0001) (FIG. 3).

The impact of moesin level of expression on overall survival of patients with breast cancers was analyzed using the online Kaplan-Meier plotter (http://kmplot.com/analysis/) for breast cancers. Patients were divided into two groups according to lower quartile of moesin expression levels. Analyses were either restricted to HER2 positive status (HER2+ by Immuno-histo chemistry (IHC) on molecular subtype HER2+ER), FIG. 2A) or restricted to HER2− breast cancer patients (FIG. 2B).

Results

Analysis conducted in vitro on different breast cancer cell lines indicated a strong inverse correlation between expression/activation levels of HER2 and expression levels of moesin (FIG. 1).

This is also found in vivo, while analyzing tumor samples from 529 patients with breast invasive carcinoma it is observed clearly that Moesin expression is low in patients with high HER2 expression (FIG. 3). Spearman correlation test indeed revealed a strong and highly significant inverse correlation between mRNA expression levels of HER2 and Moesin (r=−0.3012, P<0.0001) (FIG. 3).

Kaplan-Meier survival analysis on breast cancer data revealed that low moesin expression levels among 30 patients diagnosed with a HER2+ breast cancer were associated with a significantly shorter overall survival and a bad prognosis compared to patients with high Moesin expression (42 vs. >150 months, P=0.0239; 75% death compared to <30%) (FIG. 2A). However, this was specific of HER2+ breast cancers since level of Moesin expression did not alter survival profile of HER2− breast cancer patients (FIG. 2B). Analyzing data from METABRIC cohort supports these findings. Low moesin expression levels among 236 patients diagnosed with a HER2+ breast cancer are found associated with a significantly shorter overall survival and a bad prognosis compared to patients with high moesin expression (85.3 vs. 120.1 months, P=0.0173; 68% death compared to 55%) (FIG. 4A). Importantly, this is specific of HER2+ breast cancers since level of moesin expression does not allow to segregate HER2− breast cancer patients according their overall survival expectancy (1168 Her2− patients P=0.3676, FIG. 4B).

### CONCLUSIONS

These results demonstrate an inverse correlation between HER2 and MSN repression in vitro (in breast cancers cells) and in vivo (in tumor samples from breast cancer patients). Moreover, survival analyses demonstrated that low expression levels of moesin were associated with a shorter overall survival specifically among patients diagnosed with a HER2+ breast cancer. Therefore, moesin is a predictive biomarker for diagnosing and/or prognosticating HER2+ breast cancer in a subject. Furthermore, the datas show that the loss of MSN is specific to HER2+ BC. The loss of MSN in a patient with BC is indicative of HER2+ cancer.

1. A method for diagnosing and/or prognosticating HER2-dependent cancer in a subject, comprising:
a) measuring the amount of moesin in a sample from the subject;
b) comparing the amount of moesin measured in step a) to a reference value;
c) finding a deviation or no deviation of the amount of moesin measured in step a) from the reference value; and
d) attributing said finding of deviation or no deviation to a particular diagnosis and/or prognosis of HER2-dependent cancer in the subject.

2. The method according to claim 1, wherein a lower amount of moesin in the sample from the subject compared to the reference value indicates that the subject has HER2-dependent cancer and/or indicates a short-term survival of said subject.

3. The method according to claim 1, wherein a higher amount of moesin in the sample from the subject compared to the reference value indicates that the subject has no HER2-dependent cancer or indicates a long-term survival of said subject.

4. The method according to claim 1, wherein the sample is a tumor sample or a body fluid sample.

5. The method according to claim 4, wherein the body fluid sample is selected from the group consisting of blood, serum plasma, saliva, urine, amniotic fluid, breast milk, bronchial lavage, cerebrospinal fluid, peritoneal fluid, seminal fluid, tears and pleural fluid, preferably blood.

6. The method according to claim 2, wherein the amount of the moesin in the sample from the subject is at least 0.5 fold decreased compared to the reference value, more preferably at least 0.7 fold decreased.

7. The method according to claim 3, wherein the amount of moesin in the sample from the subject is at least 1.5 fold increased compared to the reference value, more preferably at least 2 fold increased.

8. The method according to claim 1, wherein the amount of moesin in the sample from the subject is equal to the reference value.

9. The method according to claim 1, wherein the reference value represents (i) a diagnosis of no HER2-dependent cancer or (ii) a diagnosis of HER2-dependent cancer, (iii) a good prognosis of HER2-dependent cancer or (iv) a bad prognosis of HER2-dependent cancer.

10. The method according to claim 1, wherein the HER2-dependent cancer is selected from the group consisting of breast cancer, female genital tract cancer, such as endometrial cancer, uterine cancer or ovarian cancer, bladder cancer, anal cancer, colorectal cancer, in particular uterine serous cancer, such as uterine papillary serous carcinoma, lung cancer, in particular non-small-cell lung cancer, liver cancer, kidney cancer, gastroesophageal cancer, stomach cancer, pancreas cancer and gastric cancer.

11. The method according to claim 1, wherein said HER2-dependent cancer is HER2+ breast cancer.

12. A method for monitoring a change in the diagnosis and/or prognosis of HER2-dependent cancer in a subject, comprising:
a) applying the method of claim 1 to the subject at one or more successive time points, whereby the diagnosis and/or prognosis of HER2-dependent cancer in the subject is determined at said successive time points;
b) comparing the diagnosis and/or prognosis of HER2-dependent cancer in the subject at said successive time points as determined in step a); and
c) finding the presence or absence of a change between the diagnosis of HER2-dependent cancer in the subject at said successive time points as determined in step a).

13. The method according to claim 12, wherein said change in the diagnosis and/or prognosis of HER2-dependent cancer in the subject is monitored in the course of a medical treatment of said subject.

14. The method according to claim 12, wherein the medical treatment is a prophylactic treatment or a therapeutic treatment, preferably an antitumoral treatment.

15. Use of a kit comprising:
means for measuring the amount of moesin in the sample from the subject; and
a reference value of the amount of moesin or means for establishing said reference value, said reference value representing a known diagnosis and/or prognosis of HER2-dependent cancer
for determining the diagnostic and/or the prognosis of HER2-dependent cancer in a subject suffering thereof.
